(19) United States
(12) Patent Application Publication
(10) Pub. No.: US 2009/0325159 A1
(43) Pub. Date: Dec. 31, 2009

(54) SYSTEM AND METHOD TO PREVENT CROSS-CONTAMINATION IN ASSAYS PERFORMED IN A MICROFLUIDIC CHANNEL

(75) Inventor: Shulin Zeng, Gaithersburg, MD (US)

Correspondence Address: ROTHWELL, FIGG, ERNST & MANBECK, P.C., 1425 K STREET, N.W., SUITE 800, WASHINGTON, DC, 20005, US

(73) Assignee: CANON U.S. LIFE SCIENCES, INC., Rockville, MD (US)

(21) Appl. No.: 12/164,986

(22) Filed: Jun. 30, 2008

(51) Int. Cl. C12Q 1/68; C12M 1/00; B01L 3/00

(52) U.S. Cl. 435/6; 435/285.1; 435/288.5

(57) ABSTRACT

The present application discloses systems and methods for preventing contamination in assays performed in microfluidic channels. In one embodiment, a buffer of non-reactive fluid is provided between an input port and a microchannel in which assays are performed during such times that flow from the input port is stopped. In general, an amount of non-reactive fluid is drawn into a channel connecting the stopped input port to the microchannel. Thus, any seepage, or diffusion, from the channel connecting the stopped input port to the microchannel will be of the non-reactive fluid, not the reagent, or other potentially-contaminating fluid, introduced through the input port. In one embodiment, microvalves and a negative pressure differential source control flow of reagents into the microchannel and the flow of non-reactive fluid into the inlet conduits.

### BACKGROUND

1. Field of the Invention

This invention relates to systems and methods for performing microfluidic assays. More specifically, the invention relates to systems and methods for preventing undesired materials to contaminate an assay performed in a microfluidic channel.

2. Discussion of Background

The detection of nucleic acids is central to medicine, forensic science, industrial processing, crop and animal breeding, and many other fields. The ability to detect disease conditions (e.g., cancer), infectious organisms (e.g., HIV), genetic lineage, genetic markers, and the like, is ubiquitous technology for disease diagnosis and prognosis, marker assisted selection, correct identification of crime scene features, the ability to propagate industrial organisms and many other techniques. Determination of the integrity of a nucleic acid of interest can be relevant to the pathology of an infection or cancer. One of the most powerful and basic technologies to detect small quantities of nucleic acids is to replicate some or all of a nucleic acid sequence many times, and then analyze the amplification products. Polymerase chain reaction (“PCR”) is perhaps the most well known of a number of different amplification techniques.

PCR is a powerful technique for amplifying short sections of DNA. With PCR, one can quickly produce millions of copies of DNA starting from a single template DNA molecule. PCR includes a three phase temperature cycle of denaturation of DNA into single strands, annealing of primers to the denatured strands, and extension of the primers by a thermostable DNA polymerase enzyme. This cycle is repeated so that there are enough copies to be detected and analyzed. In principle, each cycle of PCR could double the number of copies. In practice, the multiplication achieved after each cycle is always less than 2. Furthermore, as PCR cycling continues, the buildup of amplified DNA products eventually ceases as the concentrations of required reactants diminish. For general details concerning PCR, see Sambrook and Russell, Molecular Cloning—A Laboratory Manual (3rd Ed.), Vols. 1-3, Cold Spring Harbor Laboratory, Cold Spring Harbor, N.Y. (2000); Current Protocols in Molecular Biology, F. M. Ausubel et al., eds., Current Protocols, a joint venture between Greene Publishing Associates, Inc. and John Wiley & Sons, Inc., (supplemented through 2005) and PCR Protocols A Guide to Methods and Applications, M. A. Innis et al., eds., Academic Press Inc. San Diego, Calif. (1990).

Real-time PCR refers to a growing set of techniques in which one measures the buildup of amplified DNA products as the reaction progresses, typically once per PCR cycle. Monitoring the accumulation of products over time allows one to determine the efficiency of the reaction, as well as to estimate the initial concentration of DNA template molecules. For general details concerning real-time PCR see Real-Time PCR: An Essential Guide, K. Edwards et al., eds., Horizon Bioscience, Norwich, U.K. (2004).

Several different real-time detection chemistries now exist to indicate the presence of amplified DNA. Most of these depend upon fluorescence indicators that change properties as a result of the PCR process. Among these detection chemistries are DNA binding dyes (such as SYBR® Green) that increase fluorescence efficiency upon binding to double stranded DNA. Other real-time detection chemistries utilize Foerster resonance energy transfer (FRET), a phenomenon by which the fluorescence efficiency of a dye is strongly dependent on its proximity to another light absorbing moiety or quencher. These dyes and quenchers are typically attached to a DNA sequence-specific probe or primer. Among the FRET-based detection chemistries are hydrolysis probes and conformation probes. Hydrolysis probes (such as the TaqMan probe) use the polymerase enzyme to cleave a reporter dye molecule from a quencher dye molecule attached to an oligonucleotide probe. Conformation probes (such as molecular beacons) utilize a dye attached to an oligonucleotide, whose fluorescence emission changes upon the conformational change of the oligonucleotide hybridizing to the target DNA.

Commonly-assigned, co-pending U.S. application Ser. No. 11/505,358, entitled “Real-Time PCR in Micro-Channels,” the disclosure of which is hereby incorporated by reference, describes a process for performing PCR within discrete droplets flowing through a microchannel and separated from one another by droplets of non-reacting fluids, such as buffer solution, known as flow markers.

Devices for performing in-line assays, such as PCR, within microchannels include microfluidic chips having one or more microchannels formed within the chip are known in the art. These chips utilize a sample sipper tube and open ports on the chip topside to receive and deliver reagents and sample material (e.g., DNA) to the microchannels within the chip. The chip platform is designed to receive reagents at the open ports—typically dispensed by a pipetter—on the chip top, and reagent flows from the open port into the microchannels, typically under the influence of a vacuum applied at an opposite end of each microchannel. The DNA sample is supplied to the microchannel from the ports of a micro-port plate via the sipper tube, which extends below the chip and through which sample material is drawn from the ports due to the vacuum applied to the microchannel.

In some applications, it will be desirable that fluids from all of the top-side open ports flow into the microchannel, and, in other applications, it will be desirable that fluid flow from one or more, but less than all, of the top-side open ports. Also, to introduce different reagents into the microchannel via a sipper tube—typically extending down below the microchip—it is necessary to move the sipper tube from reagent container to reagent container in a sequence corresponding to the desired sequence for introducing the reagents into the microchannel. This requires that the processing instrument for performing in-line assays within the microfluidic channel of a microchip include means for effecting relative movement between the sipper tube and the different reagent containers. In addition, sipper tubes, which project laterally from a microchannel, are extremely fragile, thereby necessitating special handling, packaging, and shipping.

Furthermore, a microchip may be configured such that two or more fluid-introduction ports communicate with a common microchannel within which the assay procedure will be performed. Where more than one fluid-introduction port communicates with the microchannel and there are no valves or other devices within the microchip to physically block the port from the microchannel, it is possible that fluid from a nominally “shut off” port could seep (or diffuse) into the microchannel. This seepage or diffusion could potentially contaminate one or more assays performed in the microchannel. Flow regulation mechanisms for microchannels are therefore needed.

### SUMMARY

The present invention encompasses systems and methods for providing a buffer of non-reactive fluid between an input port and a microchannel in which assays are performed during such times that flow from the input port is stopped. In general, an amount of non-reactive fluid is drawn into a channel connecting the stopped input port to the microchannel. Thus, any seepage, or diffusion, from the channel connecting the stopped input port to the microchannel will be of the non-reactive fluid, not the reagent, or other potentially-contaminating fluid, introduced through the input port.

Aspects of the present invention are embodied in a method for preventing contamination within a microfluidic circuit which includes at least one inlet port through which fluid is introduced into the circuit, a non-reactive fluid port through which non-reactive fluid is introduced into the circuit, at least one microchannel in fluid communication with the inlet port and the non-reactive fluid port, an outlet port in fluid communication with the microchannel, and an inlet channel connecting the inlet port to the microchannel. Fluid flow into the microchannel from the inlet port is caused by applying a negative pressure differential to the outlet port and opening the inlet port to a second, higher pressure, such as atmospheric pressure, and non-reactive fluid flow into the microchannel is prevented by closing the non-reactive fluid port to the second pressure. Next, fluid flow from the inlet port is substantially stopped by closing the inlet port off to the second pressure and applying a negative pressure differential to the inlet port for a period of time to equalize the pressure between the inlet port and the inlet of the microchannel, and then shutting it off. Finally, non-reactive fluid flow into the inlet channel from the non-reactive fluid port is caused by opening the non-reactive fluid port to the second pressure, removing the negative pressure differential from the outlet port, and applying the negative pressure differential to the inlet port for a period of time to equalize the pressure between the inlet port and the inlet of the microchannel, and then shutting it off.

Other aspects of the invention are embodied in a system for preventing contamination in a microfluidic circuit. The system comprises a microfluidic circuit including at least one inlet port through which fluid is introduced into the circuit, a non-reactive fluid port through which non-reactive fluid is introduced into the circuit, at least one microchannel for fluid flow in fluid communication with the inlet port and the non-reactive fluid port, an outlet port in fluid communication with the microchannel, and an inlet channel connecting the inlet port to the microchannel. The system further includes at least one negative pressure differential source constructed and arranged for selective communication with the outlet port and the inlet port. An inlet valve mechanism is operatively associated with each inlet port and is in communication with the negative pressure differential source. The inlet valve mechanism is constructed and arranged to (1) selectively open the inlet port to a second, higher pressure, such as atmospheric pressure, while closing off the inlet port from the negative pressure differential source or (2) open the inlet port to the negative pressure differential source while closing off the inlet port to the second pressure, or (3) shut off the inlet port to maintain an established equilibrium pressure. An outlet valve mechanism is operatively associated with the outlet port and is in communication with the negative pressure differential source. The outlet valve mechanism is constructed and arranged to (1) selectively open the outlet port to the negative pressure differential source or (2) close off the outlet port to the negative pressure differential source, or (3) shut off the outlet port to maintain an established equilibrium pressure. A non-reactive fluid valve mechanism is operatively associated with the non-reactive fluid port and is constructed and arranged to (1) selectively open the non-reactive fluid port to atmospheric pressure, or the second pressure, or (2) close the non-reactive fluid port to atmospheric pressure, or the second pressure, or (3) shut off the non-reactive fluid port to maintain equilibrium attained.

According to other aspects of the invention, the system includes a controller adapted to cause fluid to flow from the inlet port into the microchannel by (1) causing the outlet valve mechanism to open the outlet port to the negative pressure differential source, (2) causing the inlet valve mechanism to open the inlet valve to atmosphere, and (3) causing the non-reactive fluid valve mechanism to close the non-reactive fluid port to atmosphere.

According to other aspects of the invention, the controller is further adapted to cause non-reactive fluid flow into the inlet channel by (1) causing the non-reactive fluid valve mechanism to open the non-reactive fluid port to atmosphere, (2) causing the outlet valve mechanism to close off said outlet port to the negative pressure differential source, and (3) causing the inlet valve mechanism to close off the inlet port to atmosphere and to open the inlet port to the negative pressure differential source.

The above and other aspects and embodiments of the present invention are described below with reference to the accompanying drawings.

### BRIEF DESCRIPTION OF THE DRAWINGS

The accompanying drawings, which are incorporated herein and form part of the specification, illustrate various embodiments of the present invention. In the drawings, like reference numbers indicate identical or functionally similar elements.

FIG. 1 is a schematic representation of a microfluidic chip and flow control system embodying aspects of the present invention.

FIG. 2 is a schematic representation of another embodiment of a microfluidic chip and flow control system embodying aspects of the present invention.

FIG. 3 is a schematic of a second alternative embodiment of a microfluidic chip and flow control system embodying aspects of the present invention.

FIG. 4 is a flow chart illustrating steps of performing a sequential, multiplex assay within a microchannel in accordance with aspects of the present invention.

FIG. 5 shows time history profiles of the flows of DNA, polymerase, assay primers, and the resulting sample test stream within a microchannel.

FIG. 6 shows time history profiles of intermittent application of negative pressure and atmospheric pressure to a fluid input well of a microfluidic chip to achieve flow metering.

FIG. 7 is a schematic representation of fluid inlet conduits interconnected with a microchannel, with flow from one of the inlet conduits into the microchannel and flow stopped in the other inlet conduits.

FIG. 8 is a schematic representation of a microfluidic chip with a non-reactive fluid inlet well and flow control system embodying aspects of the present invention.

FIG. 9 is a schematic representation of fluid inlet conduits and a non-reactive fluid inlet conduit interconnected with a microchannel, with an amount of non-reactive fluid in each conduit at its interface with the microchannel.

FIG. 10 is a schematic representation of fluid inlet conduits and a non-reactive fluid inlet conduit interconnected with a microchannel, with an amount of non-reactive fluid in all but one of the conduits at each conduit's interface with the microchannel and with fluid flow from one of the inlet conduits into the microchannel.

FIG. 11 is a flow chart showing steps for drawing non-reactive fluid from a non-reactive fluid inlet well into reactive fluid inlet conduits.

### DETAILED DESCRIPTION OF PREFERRED EMBODIMENTS

As used herein, the words “a” and “an” mean “one or more.” Furthermore, unless defined otherwise, all technical and scientific terms used herein have the same meaning as commonly understood by one of ordinary skill in the art to which the invention pertains. Although any methods and materials similar or equivalent to those described herein can be used in the practice of the present invention, the preferred materials and methods are described herein.

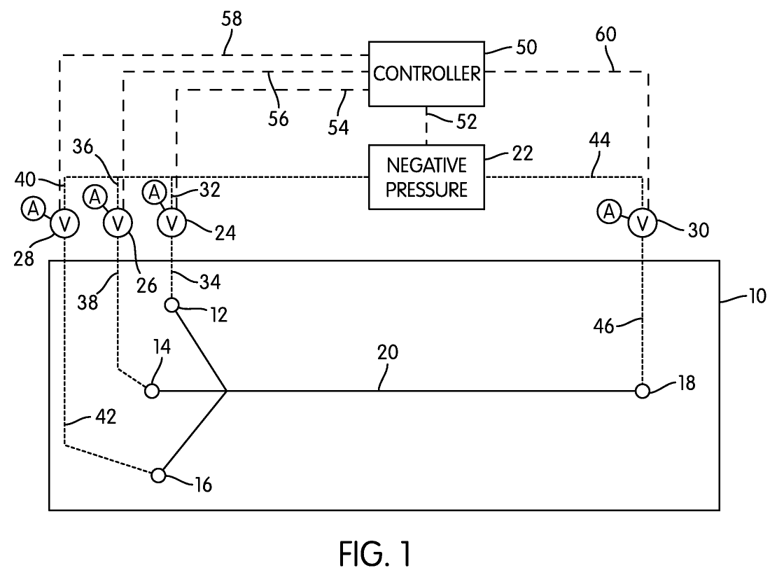

A system for microfluidic flow embodying aspects of the present invention is shown in FIG. 1. The system includes a microfluidic circuit which, in the illustrated embodiment, is carried on a microfluidic chip 10. Microfluidic chip 10 includes inlet ports 12, 14, 16, a microchannel 20 that is in fluid communication with the inlet ports 12, 14, 16, and an outlet port 18 also in fluid communication with the microchannel 20. The embodiment shown in FIG. 1 is exemplary; the microfluidic circuit may include more or less than three inlet ports and may include more than one microchannel in communication with some or all of the inlet ports. The microfluidic circuit may also include more than one outlet port. Fluid is introduced into the circuit through the fluid inlet ports 12, 14, and 16. Fluid may be provided to the fluid inlet ports in any appropriate manner known in the art. Or, alternatively, fluid may be provided to the fluid inlet ports by means of a fluid-containing cartridge coupled to each port in a fluid-communicating manner as described in commonly assigned U.S. patent application Ser. No. 11/850,229 “Chip and cartridge design configuration for performing micro-fluidic assays”, the disclosure of which is hereby incorporated by reference.

Fluid is collected from the microchannel 20 through the fluid outlet 18 and may be deposited in any appropriate waste reservoir, such as, for example, a chip as described in the commonly assigned U.S. patent application Ser. No. 11/850,229.

The microfluidic chip 10 may be formed from glass, silica, quartz, or plastic or any other suitable material.

Fluid movement through the circuit is generated and controlled by means of a negative pressure differential applied between the outlet port 18 and one or more of the inlet ports 12, 14, 16. Application of a negative pressure differential between the outlet port 18 and one or more of the inlet ports 12, 14, 16 will cause fluid flow from the inlet port(s), through the microchannel 20 and to the outlet port 18. A pressure differential can be generated by one or more pressure sources, such as negative pressure source 22, which, in one embodiment, may comprise a vacuum pump. In the illustrated embodiment, pressure differentials between the outlet port 18 and the inlet ports 12, 14, 16 is controlled by means of pressure control valves controlling pressure at each of the inlet ports 12, 14, 16 and the outlet port 18.

More specifically, a pressure control valve 30 is arranged in communication with the pressure source 22 and the outlet port 18. Similarly, a pressure control valve 24 is arranged in communication with the inlet port 12, a pressure control valve 26 is arranged in communication with the inlet port 14, and a pressure control valve 28 is arranged in communication with the inlet port 16. Arrangements having more than three inlet ports would preferably have a pressure control valve associated with each inlet port. In the illustrated embodiment of FIG. 1, valves 24, 26, 28 are three-way valves which may selectively connect each associated inlet port 12, 14, 16, respectively, to either atmospheric pressure, represented by the circled letter “A”, or an alternative pressure source, which may be the negative pressure source 22. That is, in the illustrated embodiment, valve 24 is in communication pressure source 22 via pressure line 32 and is in communication with inlet port 12 via pressure line 34. Valve 26 is in communication with pressure source 22 via pressure line 36 and is in communication with inlet port 14 via pressure line 38. Valve 28 is in communication with pressure source 22 via pressure line 40 and is in communication with inlet port 16 via pressure line 42. Valve 30 is connected via pressure line 44 to the pressure source 22 and by pressure line 46 to outlet port 18. In the illustrated embodiment, valve 30 is also a three-way valve for selectively connecting the outlet port 18 to either atmospheric pressure, indicated by the circled “A”, or to the pressure source 22.

Pressure source 22 and valves 24, 26, 28, 30 may be controlled by a controller 50. Controller 50 is connected via a control line 52 to the pressure source 22, via a control line 54 to the valve 24, via a control line 56 to valve 26, via a control line 58 to valve 28, and via a control line 60 to valve 30. Controller 50 may also be connected to one or more of the various components wirelessly or by other means known to persons of ordinary skill in the art. Controller 50 may comprise a programmed computer or other microprocessor.

As mentioned above, fluid flow from an inlet port 12, 14, and/or 16 through the microchannel 20 and to the outlet port 18 is generated by the application of a negative pressure differential between the outlet port 18 and one or more of the inlet ports. More specifically, to generate a fluid flow from inlet port 12, a negative pressure is applied to the outlet port 18 by connecting the negative pressure source 22 to the outlet port 18 via the control valve 30 and pressure lines 44 and 46. Inlet port 12 is opened to atmospheric pressure by valve 24. This creates the negative pressure differential between the outlet port 18 and the inlet port 12. Assuming that fluid flow from other inlet ports is not desired while fluid is flowing from the inlet port 12, inlet port 14 is closed to atmospheric pressure by valve 26 and inlet port 16 is closed to atmospheric pressure by valve 28. To stop fluid flow from the inlet port 12, valve 24 is activated (e.g., via the controller 50) to close off the inlet port 12 to atmospheric pressure. To rapidly stop the flow of fluid from the inlet port 12, it may be desirable to connect the inlet port 12 to the negative pressure source 22 via the control valve 24 for a period of time sufficient to equalize the pressure between the inlet port 12 and the inlet of the microchannel, and then to shut off control valve 24.

A predetermined volume of fluid can be introduced into the microchannel 20 from any of the inlet ports 12, 14, and 16—assuming the flow rate generated by the pressure differential between the outlet port 18 and the applicable inlet port is known—by maintaining the pressure differential for a period of time which, for the generated flow rate, will introduce the desired volume of fluid into the microchannel 20. Maintaining the pressure differential can be effected by proper control of the pressure control valves associated with the inlet ports and the outlet port.

Activation and timing of the control valve 24 may be controlled by the controller 50.

To then generate fluid flow from the inlet port 14, valve 26 is activated (e.g., by controller 50) to open inlet port 14 to atmospheric pressure while negative pressure is applied to the outlet port 18, thus creating the negative pressure differential between the outlet port 18 and the inlet port 14. Fluid flow from the inlet port 14 is stopped by activating valve 26 to close inlet port 14 to atmospheric pressure, and, to rapidly stop flow from the inlet port 14, valve 26 opens the inlet port 14 to the negative pressure source 22 for a period of time sufficient to equalize the pressure between the inlet of the microchannel and the inlet port 14, and then shut off valve 26.

Similarly, to generate fluid flow from the inlet port 16, valve 28 is activated (e.g., by controller 50) to open inlet port 16 to atmospheric pressure while negative pressure is applied to the outlet port 18, thus creating the negative pressure differential between the outlet port 18 and the inlet port 16. Fluid flow from the inlet port 16 is stopped by activating valve 28 to close inlet port 16 to atmospheric pressure, and, to rapidly stop flow from the inlet port 16, valve 28 opens the inlet port 16 to the negative pressure source 22 for a period of time sufficient to equalize the pressure between the inlet of the microchannel and the inlet port 16, and then shut off valve 28.

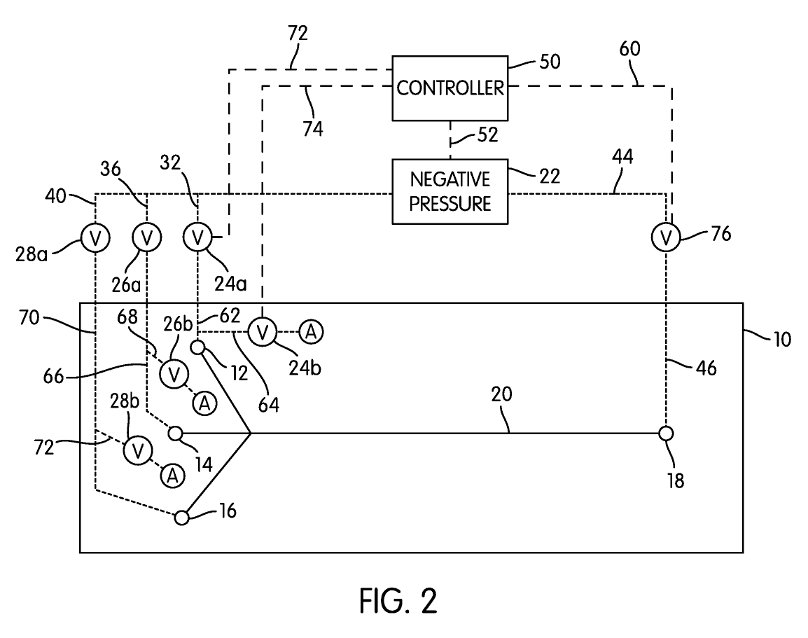

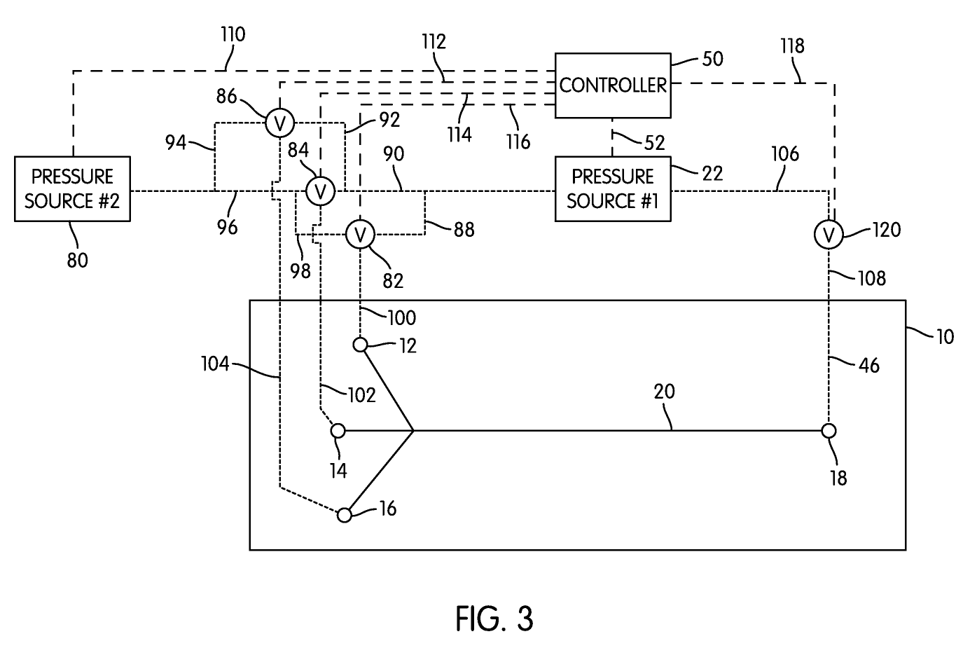

FIGS. 2 and 3 show alternative arrangements for controlling the pressure differential between an outlet port and one or more of the inlet ports of a microfluidic circuit. FIG. 2 shows a system similar to that shown in FIG. 1 except that each inlet port 12, 14, 16 is coupled to two two-way valves as opposed to a single three-way valve. More specifically, inlet port 12 is coupled to a first two-way valve 24a for selectively connecting the inlet port 12 to the pressure source 22 via pressure lines 32 and 62. Inlet port 12 is also coupled to a second two-way valve 24b for selectively connecting the inlet port 12 to atmospheric pressure “A” via pressure line 64.

Similarly, inlet port 14 is coupled to a first two-way valve 26a for selectively connecting port 14 to the pressure source 22 via pressure lines 36 and 66 and to a second two-way valve 26b for selectively connecting the inlet port 14 to atmospheric pressure via pressure line 68. Inlet port 16 is coupled to a first two-way valve 28a for selectively connecting the inlet port 16 to the pressure source 22 via pressure lines 40 and 70 and to a second two-way valve 28b for selectively connecting the inlet port 16 to atmospheric pressure via pressure line 72.

In the system shown in FIG. 2, outlet port 18 is coupled to two-way valve 76 for selectively connecting the outlet port 18 to the pressure source 22 via pressure lines 44 and 46.

Controller 50 controls the negative pressure source 22 via control line 52, controls two-way valve 76 via control line 60, controls two-way valve 24a via control line 72, and controls two-way valve 24b via control line 74. Controller 50 is also linked to valves 26a, 26b, 28a, and 28b for controlling those valves, but the control connections between the controller 50 and the respective valves are not shown in FIG. 2 so as to avoid unnecessarily cluttering the Figure.

FIG. 3 shows an alternative arrangement of the system embodying aspects of the present invention. In the embodiment of FIG. 3, each inlet port 12, 14, 16 is coupled to a three-way valve for selectively connecting the port either to pressure source #1 22, or pressure source #2 80. More specifically, inlet port 12 is coupled to valve 82 configured to selectively connect the inlet port 12 to pressure source #1 22 via pressure lines 88, 90, and 100 or to pressure source #2 80 via pressure lines 96, 98, and 100. Inlet port 14 is coupled to valve 84 configured to selectively connect inlet port 14 to the pressure source #1 22 via pressure lines 90 and 102 or to pressure source #2 80 via pressure lines 96 and 102. Inlet port 16 is coupled to pressure valve 86 configured to selectively couple port 16 to pressure source #1 22 via pressure lines 90, 92 and 104 or to pressure source #2 80 via pressure lines 96, 94 and 104. Outlet port 18 is coupled to valve 120 for selectively connecting outlet port 18 to pressure source #1 22 via pressure lines 106 and 46.

Controller 50 controls pressure source #1 22 via control line 52 and controls pressure source #2 80 via control line 110. Controller 50 also controls pressure control valve 120 via control line 118, pressure valve 82 via control line 116, pressure valve 84 via control line 114, and pressure valve 86 via control line 112.

To generate fluid flow from inlet port 12, control valve 120 is activated (e.g., by controller 50) to connect outlet port 18 to pressure source #1 22, and control valve 82 is activated to connect inlet port 12 to pressure source #2 80. The pressure generated by pressure source #2 80 is preferably greater than the pressure generated by pressure source #1 22. Thus, a negative pressure differential is created between outlet port 18 and inlet port 12. Inlet ports 14 and 16 are initially connected, by valves 84 and 86, respectively, to pressure source #1 22, so there is no pressure differential between inlet ports 14 and 16 and the inlet of the microchannel and thus no fluid flow from inlet ports 14 and 16 to outlet port 18. Valves 84 and 86 may be shut off to maintain the established equilibrium pressures. To stop fluid flow from inlet port 12, control valve 82 is activated to connect inlet port 12 to pressure source #1 22 to equalize the pressure between the inlet of the microchannel and the inlet port 12, and then shut off control valve 82.

To generate fluid flow from inlet port 14, control valve 84 is activated to connect inlet port 14 to pressure source #2 80 to create a negative pressure differential between outlet port 18 and inlet port 14. Valves 82 and 86 to inlet ports 12 and 16 are shut off to maintain established pressures, so there is no pressure differential between inlet ports 12 and 16 and the inlets of the microchannel, and thus no fluid flow from inlet ports 12 and 16 to outlet port 18. To stop fluid flow from inlet port 14, control valve 84 is activated to connect inlet port 14 to pressure source #1 22 to equalize the pressure between the inlet of the microchannel and the inlet port 14, and then shut off valve 84.

To generate fluid flow from inlet port 16, control valve 86 is activated to connect inlet port 16 to pressure source #2 80 to create a negative pressure differential between outlet port 18 and inlet port 16. Valves 82 and 88 to inlet ports 12 and 14 are shut off to maintain established pressures, so there is no pressure differential between inlet ports 12 and 14 and inlets of the microchannel, and thus no fluid flow from inlet ports 12 and 14 to outlet port 18. To stop fluid flow from inlet port 16, control valve 86 is activated to connect inlet port 16 to pressure source #1 22 to equalize the pressure between the inlet of the microchannel and the inlet port 16, and then shut off valve 86.

As an alternative arrangement, three-way valves 82, 84, 86 could each be replaced by two two-way valves for selectively connecting each associated inlet port with pressure source #1 22 or pressure source #2 80.

Suitable valves for use in the present invention include two-way and three-way solenoid valves by IQ Valves Co., Melbourne, Fla. and The Lee Company, Westbrook, Conn.

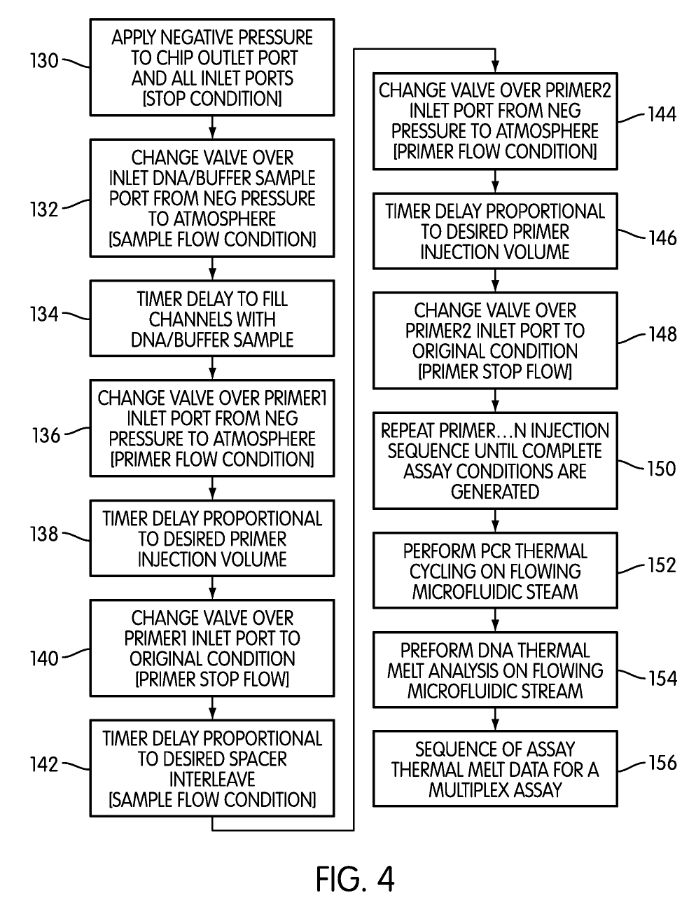

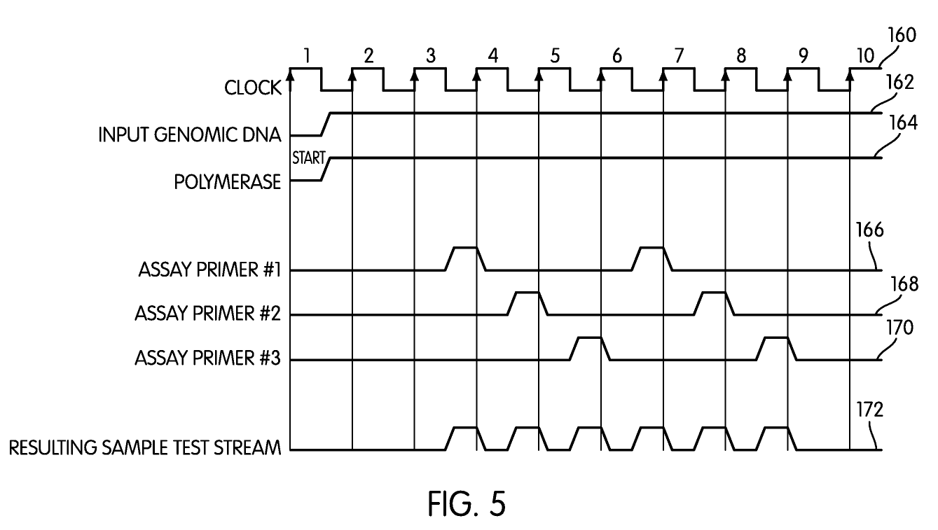

The systems shown in FIGS. 1, 2 and 3 can be utilized in a process for performing PCR within discreet droplets of assay reagents flowing through a microchannel and separated from one another by droplets of non-reacting fluids, such as buffer solution, as is described in commonly assigned, co-pending U.S. application Ser. No. 11/505,358. The process will be described with reference to FIGS. 4 and 5.

FIG. 4 is a flow chart illustrating the steps for performing PCR within discreet droplets flowing through a microchannel, and FIG. 5 shows time history curves representing the flow of various materials through the channel. The process will be described with reference to the system shown in FIG. 1. It should be understood, however, that the process could also be performed with the systems of FIG. 2 or 3 or a hybrid combination of the systems of FIGS. 1, 2, and 3.

Referring to FIG. 4, at step 130 negative pressure is applied to the outlet port 18 and all of the inlet ports 12, 14, 16, etc, by connecting the ports, via the associated valves, to negative pressure source 22. All inlet valves are shut off at this moment. This is known as a stop condition as there is no pressure differential between the waste port and any inlet port, and thus no fluid flow into the microchannel 20.

In step 132, the valve coupled to the DNA/buffer inlet port (e.g., valve 24 associated with inlet port 12) is switched from negative pressure to atmospheric pressure to generate a sample flow condition (i.e., a negative pressure differential between outlet port 18 and inlet port 12) as shown by the curve 162 in FIG. 5. Although not shown in FIG. 4, a valve coupled to a polymerase inlet port may also be switched from negative pressure to atmospheric pressure to generate a polymerase flow as shown by curve 164 in FIG. 5. The DNA/buffer mixture is combined into a common flow through the microchannel 20.

In step 134, a timer delay is implemented to fill the channels with the DNA/buffer (and optionally polymerase) mixture.

In step 136, the valve coupled to a PRIMER1 inlet port (e.g., valve 26 associated with inlet port 14) is switched from negative pressure to atmospheric pressure to generate a primer flow condition into the microchannel 20 to be mixed with the sample flow stream. A timer delay that is proportional to the desired timer injection volume is implemented in step 138 to control the volume of PRIMER1 that flows into the mixture. In step 140, the valve coupled to PRIMER1 inlet port is switched to the original condition, i.e., negative pressure with the valve shutting off, to stop primer flow, thereby generating the first portion of flow curve 166 (through clock interval 4) in FIG. 5.

A timer delay proportional to a desired spacer interleave is implemented in step 142. This is a sample flow condition without primer flowing.

In step 144, the valve coupled to the PRIMER2 inlet port (e.g., valve 28 associated with inlet port 16) is changed from negative pressure to atmospheric pressure to generate a primer flow condition into the microchannel 20 to be mixed with the sample flow stream. A timer delay that is proportional to the desired injection volume of PRIMER2 is implemented in step 146. And, in step 148, the valve coupled to the PRIMER2 inlet port is switched back to the original, negative pressure with a valve being in the shut off condition to stop the flow of PRIMER2. Steps 144, 146, and 148 generate the first portion of flow curve 168 (through clock interval 5) shown in FIG. 5.

In step 150, a primer injection sequence is repeated for additional primers and additional, discrete injections of previously-injected primers until the complete assay conditions are generated, thus generating flow curve 170. The resulting sample test stream flow curve is designated by curve 172 in FIG. 5 in which each “hump” in the curve represents a discrete volume of a primer mixed in the sample flow stream. A separate PCR (or other) assay can be performed in each discrete volume (or bolus) of sample/primer mixture.

In step 152, PCR thermal cycling is performed on the flowing microfluidic stream thereby generating a PCR amplification reaction within each test bolus. In step 154, a DNA thermal melt analysis is performed on the flowing microfluidic stream. And, in step 156, a sequence of assay thermal melt data is generated for each test bolus for a multiplex assay performed within the microchannel 20.

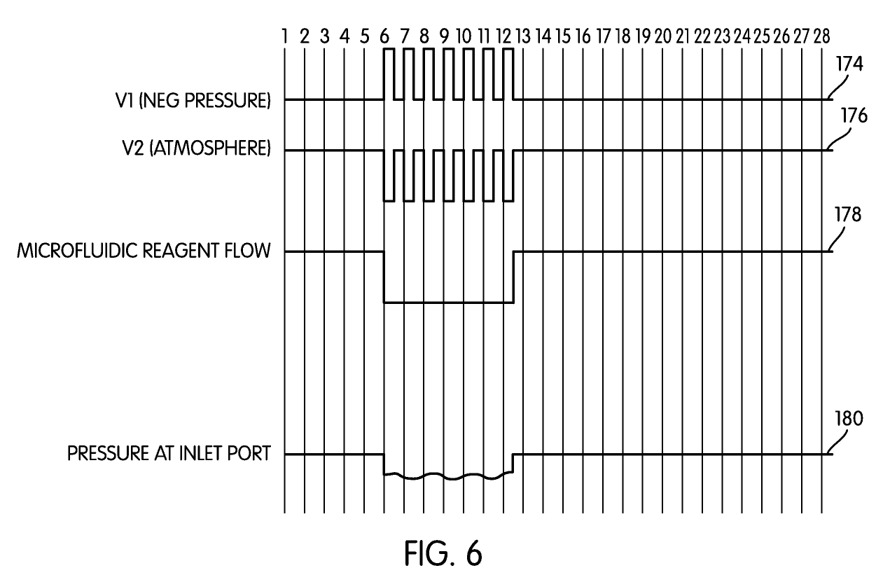

As shown in FIG. 6, any valve coupled to an inlet port can be operated in a pulse width modulated manner to regulate the volume of fluid injected at the inlet port. For example, as described above, a valve coupled to an inlet port can be set to a flow condition for a predetermined period of time corresponding to a desired volume of fluid to be injected into the microchannel. A smaller volume of fluid can be injected by having the valve coupled to the inlet port set to the flow condition for a shorter period of time. It may be desirable, however, to produce reaction droplets of a specified physical size and, thus, it may be desirable to have fluid flow from the inlet port for the specified period of time (and not the shorter time corresponding to the smaller volume). To produce a lower volume of fluid flow from an inlet port while maintaining the flow from the port for a specified period of time, the valve coupled to the port may be modulated between negative pressure and atmospheric pressure (or other higher pressure) over the desired flow period, as shown in curves 174 and 176 in FIG. 6. The resulting pressure at the inlet port is indicated by curve 180 in FIG. 6. The resulting reagent flow, as shown in curve 178 in FIG. 6, is a generally constant flow over the entire flow period at a flow rate that will result in a lower volume of fluid injected than if the inlet valve were kept open to atmospheric pressure for the entire flow period.

The systems and methods described above provide means for quickly starting and stopping fluid flow from input ports to a microfluidic channel, allowing precise volumetric control and timing of the fluid flow. When fluid flow from a particular input port is stopped, an interface is created between the fluid introduced at that port and the fluid contained within the microchannel. A small amount of fluid from the stopped input port may diffuse into the microchannel which can cause contamination if an undesired fluid is mixed with a test volume.

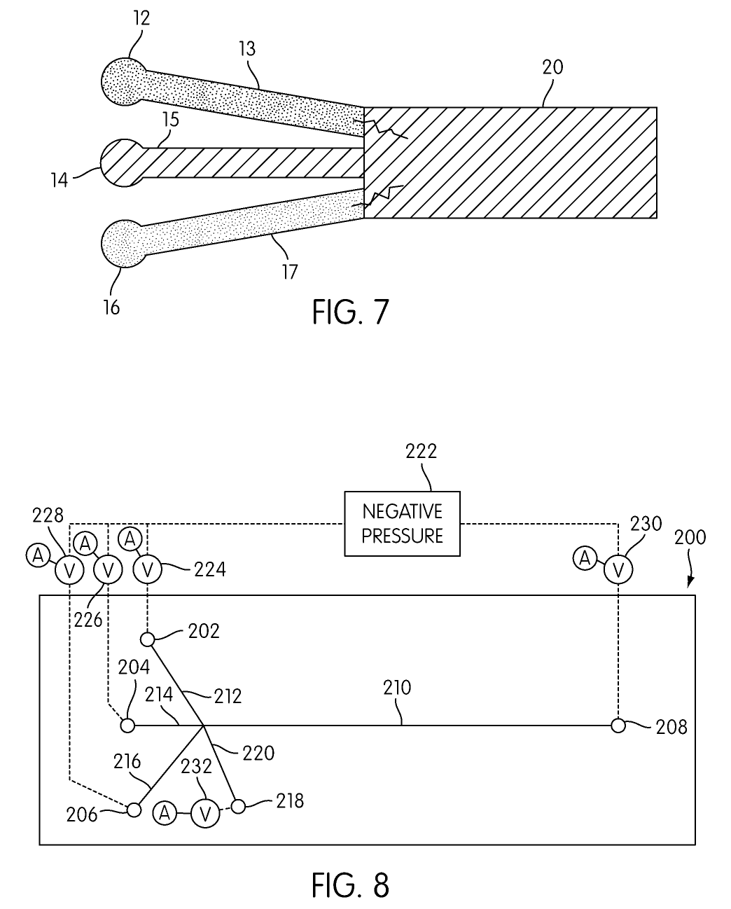

This is schematically illustrated in FIG. 7 which shows input ports 12, 14, 16 in communication with the microchannel 20 via input channels 13, 15, 17, respectively. As shown in FIG. 7, fluid is flowing from input port 14 through input channel 15 and into the microchannel 20, as represented by the crosshatching in the figure, while fluid flow from inlet ports 12 and 16 is stopped, as represented by the stippling in FIG. 7. This condition creates a fluid interface between fluid within inlet channels 13 and 17, connecting inlet ports 12 and 16, respectively, to the microchannel 20, and the fluid in the microchannel 20. An amount of fluid from the inlet channels 13 and 17 may diffuse into the microchannel 20, as represented by jagged lines extending across the fluid interface in FIG. 7.

FIG. 8 illustrates a system for alleviating the problem of fluid diffusing from inlet ports for which the flow has been stopped into the microchannel. The system shown in FIG. 8 includes a microfluidic chip 200 having an outlet port 208 in communication with a microchannel 210 and inlet ports 202, 204, 206, and 218 in communication with the microchannel 210 via inlet channels 212, 214, 216, and 220, respectively. The system further includes a negative pressure source 222, a valve 230 associated with outlet port 208, a valve 224 associated with inlet port 202, a valve 226 associated with inlet port 204, a valve 228 associated with inlet port 206, and a valve 232 associated with inlet port 218.

The system is configured such that outlet port 208 can be selectively coupled, via the valve 230, to either the negative pressure source 222 or atmospheric pressure “A”. Inlet port 202 can be selectively coupled, via valve 224, to the negative pressure source 222, or atmospheric pressure, or a negative pressure with the valve shutting off. Inlet port 204 can be selectively coupled, via valve 226, to the negative pressure source 222, or atmospheric pressure, or a negative pressure with the valve shutting off. Inlet port 206 can be selectively coupled, via valve 228, to the negative pressure source 222, or atmospheric pressure, or a negative pressure with the valve shutting off.

In the illustrated embodiment, each of the valve 230, 224, 226, 228 is a three-way valve for selectively connecting the associated port either to the negative pressure source 222, or atmospheric pressure, or a negative pressure with the valve shutting off. Alternatively, the system may be configured with two two-way valves associated with each port, one valve for selectively connecting the associated port to the negative pressure source and the other valve for selectively connecting the associated port to atmospheric pressure, for example, as shown and described in connection with FIG. 2 above. As a further alternative, the system may include a second pressure source adapted to generate pressure higher than that of the negative pressure source 222, and each port can be selectively coupled, via associated valve or valves, to either of the pressure sources, for example, as described above with respect to FIG. 3.

Control valve 232, associated with inlet port 218, may be a two-way valve for selectively connecting the inlet port 218 to atmospheric pressure for closing off the connection between inlet port 218 and atmospheric pressure.

Although not shown in FIG. 8, each of the control valves and the negative pressure source are preferably controlled by a controller.

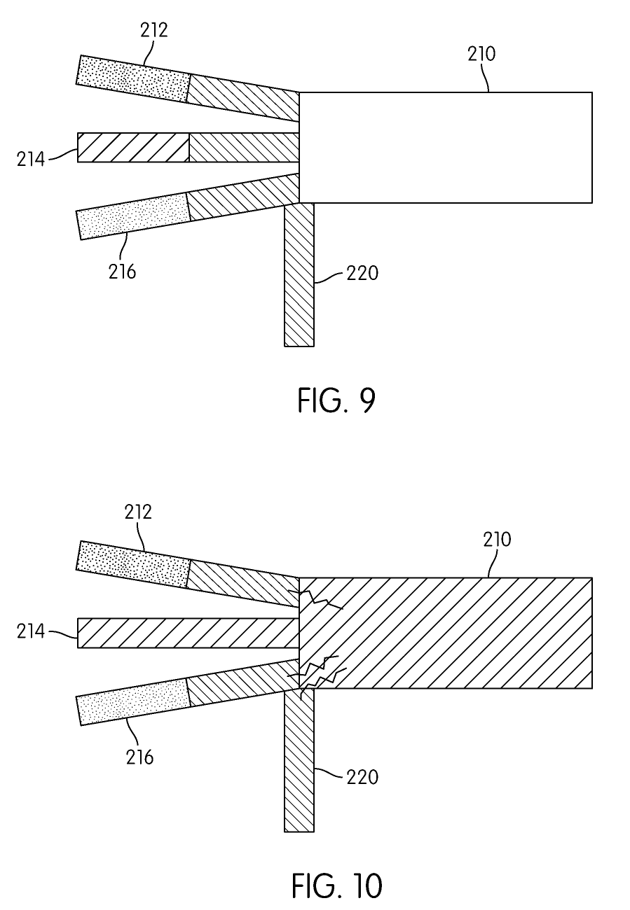

A source of nonreactive fluid (e.g., a buffer solution) is coupled to the inlet port 218. The inlet ports 202, 204, 206 (through which reactive fluids (e.g., reagents) are introduced) are coupled by their respective valves to the negative pressure source 222, while inlet port 218 is opened to atmospheric pressure by valve 232. This creates a negative pressure differential between the reagent inlet ports 202, 204, 206 and the buffer inlet port 218, thus drawing an amount of buffer solution (or other non-reactive fluid) from the inlet port 218 into the inlet channels 212, 214, 216. This is schematically represented in FIG. 9, which shows an amount of buffer solution, indicated by crosshatching, drawn from the inlet channel 220, connecting the buffer inlet port 218, partially into each of the reagent inlet channels 212, 214, 216. Thus, the fluid interface between each of the reagent inlet channels 212, 214, 216 and the microchannel 210 is merely an interface with a non-reactive buffer solution, thus avoiding the problem of reactive fluid diffusing into the microchannel at a fluid interface.

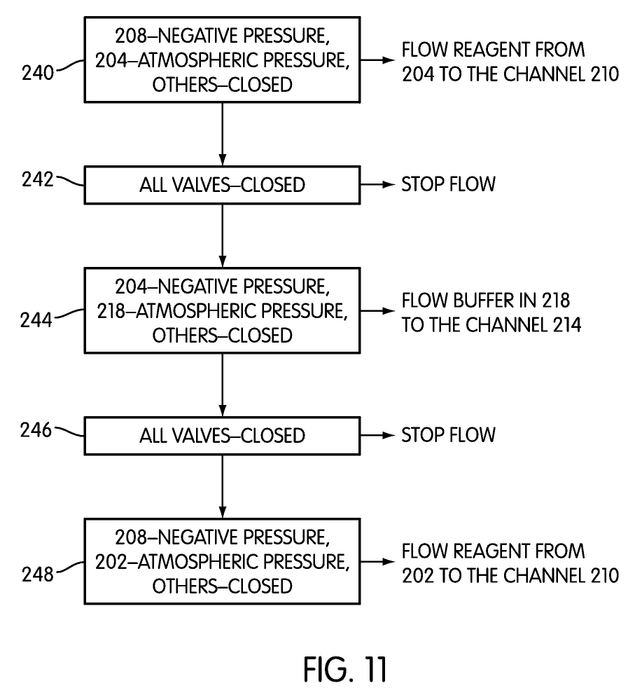

FIGS. 10 and 11 illustrate a process for generating reagent flow while avoiding diffusion-caused contamination in accordance with this aspect of the invention.

In step 240 of FIG. 11, after an amount of buffer solution has been drawn into each of the inlet channels 212, 214, 216, as shown in FIG. 9, negative pressure is applied to the outlet port 208 by connecting the outlet port 208 to the negative pressure source 222 via valve 230. Reagent inlet port 204 is open to atmospheric pressure by valve 226, thus causing reagent to flow from the reagent inlet port 204 through the inlet channel 214 and into the microchannel 210. In step 242, after injecting a predetermined volume of reagent fluid from the inlet port 204, all valves are closed, thus stopping any further flow from the inlet port 204.

In step 244, reagent inlet port 204 is opened to negative pressure source 222 by the valve 226, and buffer inlet port 218 is opened to atmospheric pressure by valve 232, thus causing buffer to flow from the inlet port 218 through the inlet channel 220 and into the inlet channel 214. This will again create a non-reactive fluid interface between inlet channel 214 and microchannel 210, shown in FIG. 9.

In step 246, after drawing a predetermined amount of buffer solution into the inlet channel 214, all valves are closed to stop any further flow. In step 248, outlet port 208 is again connected to the negative pressure source 222 by the valve 230 and reagent inlet port 202 is opened to atmospheric pressure by the valve 224 while all other valves are closed, thus causing reagent to flow from inlet port 202 into the microchannel 210.

As represented in FIG. 10, while reactive fluid is flowing from the inlet port 204 and inlet channel 214 into the microchannel 210, any diffusion from the other inlet channels 212, 216, 220 into the microchannel 210 merely involves a diffusion of buffer solution at the interface between the fluid in each inlet channel and the microchannel 210. Thus, diffusion from non-flowing inlet channels does not cause contamination of a test volume of reactive fluid introduced at inlet port 214.

The amount of buffer solution drawn into a reagent inlet channel will depend on the period of time during which flow from that channel will be stopped. For example, if flow from a particular reagent inlet channel will be stopped for a relatively long period of time, there will be more time for reagent fluid to diffuse through the buffer interface and into the microchannel, whereas if flow from the reagent inlet channel will be stopped for a relatively short time, there will be relatively less time for such diffusion to occur. Thus, the size of the buffer interface between the reagent fluid and the microchannel may depend on the amount of time that flow is stopped from that reagent inlet channel. The length of the buffer interface is preferably about 1 mm but may range from 0.2 mm up to 5 mm. If flow from a particular inlet channel will be stopped for only two minutes, a buffer interface of 0.2 mm may be sufficient, whereas if flow from a reagent inlet channel will be stopped for one hour, a buffer interface of 3-5 mm may be desirable. Longer or shorter buffer interfaces can be selected as well.

While the present invention has been described and shown in considerable detail with disclosure to certain preferred embodiments, those skilled in the art will readily appreciate other embodiments of the present invention. Accordingly, the present invention is deemed to include all modifications and variations encompassed within the spirit and scope of the following appended claims.

Additionally, while the processes described above and illustrated in the drawings are shown as a sequence of steps, this was done solely for the sake of illustration. Accordingly, it is contemplated that some steps may be added, some steps may be omitted, and the order of the steps may be re-arranged.

We claim:

1. A method for preventing contamination within a microfluidic circuit including at least one inlet port through which fluid is introduced into the circuit, a non-reactive fluid port through which non-reactive fluid is introduced into the circuit, at least one microchannel for fluid flow in fluid communication with the inlet port and the non-reactive fluid port, an outlet port in fluid communication with the microchannel through which the fluid from the microchannel are collected, and an inlet channel connecting the inlet port to the microchannel, said method comprising the steps of:
a. causing fluid flow into the microchannel from the inlet port by applying a negative pressure differential between the outlet port and the inlet port while substantially preventing non-reactive fluid from flowing from the non-reactive fluid port;
b. substantially stopping fluid flow into the microchannel from the inlet port by removing the negative pressure differential between the outlet port and the inlet port; and
c. causing non-reactive fluid flow into the inlet channel from the non-reactive fluid port by applying a negative pressure differential between the inlet port and the non-reactive fluid port.

2. The method of claim 1, wherein the step of causing fluid flow into the microchannel from the inlet port comprises applying a first pressure to the outlet port and applying a second pressure higher than the first pressure to the inlet port to generate the negative pressure differential between the outlet port and the inlet port.

3. The method of claim 2, wherein the first pressure is a negative pressure and the second pressure is atmospheric pressure.

4. The method of claim 3, wherein the step of preventing non-reactive fluid from flowing from the non-reactive fluid port comprises closing the non-reactive fluid port to atmosphere during step a.

5. The method of claim 3, wherein the stopping step comprises closing off the inlet port to atmosphere to remove the pressure differential between the outlet port and the inlet port.

6. The method of claim 1, wherein the stopping step comprises applying substantially the same pressure to the outlet port and the inlet port for a predetermined period of time, and then shutting off the valve to maintain an established negative pressure.

7. The method of claim 1, wherein the step of causing non-reactive fluid flow into the inlet channel from the non-reactive fluid port comprises applying a first pressure to the inlet port and applying a second pressure higher than the first pressure to the non-reactive fluid port.

8. The method of claim 7, wherein the first pressure is a negative pressure and the second pressure is atmospheric pressure.

9. The method of claim 6, further comprising, after the predetermined period of time, again causing fluid flow into the microchannel from the inlet port by applying the negative pressure differential between the outlet port and the inlet port.

10. The method of claim 1, wherein the microfluidic circuit comprises a plurality of inlet ports and the at least one microchannel is in fluid communication with each of the inlet ports via an associated inlet channel connecting each inlet port to the microchannel, and wherein the method further comprises, during step a, substantially preventing fluid flow from all other inlet ports by preventing a negative pressure differential between the outlet port and the other ports.

11. The method of claim 10, further comprising:
d. causing fluid flow into the microchannel from a second inlet port by applying a negative pressure differential between the outlet port and the second inlet port while substantially preventing fluid flow from all other inlet ports by preventing a negative pressure differential between the outlet port and the other ports; and then
e. substantially stopping the fluid from the second inlet port by removing the negative pressure differential between the outlet port and the second inlet port; and
f. causing non-reactive fluid flow into the second inlet channel from the non-reactive fluid port by applying a negative pressure differential between the second inlet port and the non-reactive fluid port.

12. The method of claim 10, further comprising repeating steps a through c for each of the inlet ports.

13. The method of claim 1, wherein the fluid introduced from the inlet port comprises a biological sample material, a reagent, or a marker material.

14. The method of claim 1, further comprising controlling the duration of step a to control the volume of fluid that flows from the inlet port into the microchannel by commencing step b after a predetermined duration of step a corresponding to the flow of a predetermined volume of fluid from the inlet port into the microchannel.

15. The method of claim 14, further comprising:
specifying a predetermined duration of step a corresponding to a predetermined volume of fluid flow; and
metering a volume of fluid flow from the inlet port into the microchannel that is less than the predetermined volume by alternately applying and removing the negative pressure differential between the outlet port and the inlet port during the predetermined duration.

16. The method of claim 15, wherein the metering step comprises applying a negative pressure to the outlet port and alternately (1) opening the inlet port to atmosphere and (2) closing the inlet port to atmosphere during the predetermined duration.

17. The method of claim 1, wherein the non-reactive fluid is a buffer solution.

18. The method of claim 1, wherein the amount of non-reactive fluid caused to flow into the inlet channel during step c fills the inlet channel to a length of 200 microns to 5 mm.

19. The method of claim 1, further comprising, prior to step a, causing an amount of non-reactive fluid to flow into the inlet channel from the non-reactive fluid port by applying a negative pressure between the inlet port and the non-reactive fluid port.

20. A system for preventing contamination in a microfluidic circuit comprising:
a. microfluidic circuit comprising:
i. at least one inlet port through which fluid is introduced into said circuit;
ii. a non-reactive fluid port through which non-reactive fluid is introduced into said circuit;
iii. at least one microchannel for fluid flow in fluid communication with said inlet port and said non-reactive fluid port;
iv. an outlet port in fluid communication with said microchannel through which the fluid and the non-reactive fluid from said microchannel are collected; and
v. an inlet channel connecting said inlet port to said microchannel;
b. at least one pressure source constructed and arranged for selective communication with said outlet port and said at least one inlet port;
c. an outlet valve mechanism operatively associated with said outlet port and in communication with said pressure source, said outlet valve mechanism being constructed and arranged to (1) selectively open said outlet port to a first pressure generated by said pressure source or (2) close off said outlet port to said first pressure;
d. an inlet valve mechanism operatively associated with each inlet port and in communication with said pressure source, said inlet valve mechanism being constructed and arranged to (1) selectively open said inlet port to a second pressure higher than said first pressure or (2) open said inlet port to said first pressure or be shut off to maintain an established pressure; and
e. a non-reactive fluid valve mechanism operatively associated with said non-reactive fluid port and constructed and arranged to (1) selectively open said non-reactive fluid port to said second pressure or (2) close said non-reactive fluid port to said second pressure or be shut off to maintain an established pressure.

21. The system of claim 20, wherein said at least one pressure source comprises a vacuum pump, said first pressure comprises a negative pressure generated by said vacuum pump, and said second pressure comprises atmospheric pressure.

22. The system of claim 20, wherein said at least one pressure source comprises a first pump for generating said first pressure and a second pump for generating said second pressure.

23. The system of claim 20, further comprising a controller adapted to control the operation of said outlet valve mechanism, said inlet valve mechanism, and said non-reactive fluid valve mechanism and to cause fluid to flow from said inlet port into said microchannel by causing said outlet valve mechanism to open said outlet port to said first pressure and causing said inlet valve mechanism to open said inlet valve to said second pressure to generate a negative pressure differential between said outlet port and said inlet port and to substantially prevent non-reactive fluid flow from said non-reactive fluid port by causing said non-reactive fluid valve mechanism to close said non-reactive fluid port to said second pressure.

24. The system of claim 23, wherein said controller is further adapted to stop fluid flow from said inlet port into said microchannel by causing said inlet valve mechanism to close off said inlet port to said second pressure and to open said inlet port to said first pressure and to be shut off to maintain the established pressure.

25. The system of claim 20, wherein said controller is further adapted to cause non-reactive fluid flow into said inlet channel by (1) causing said non-reactive fluid valve mechanism to open said non-reactive fluid port to said second pressure, (2) causing said outlet valve mechanism to close off said outlet port to said first pressure, and (3) causing said inlet valve mechanism to close off said inlet port to said second and to open said inlet port to said first pressure.

26. The system of claim 20, wherein said microfluidic circuit comprises:
a plurality of inlet ports;
an inlet channel associated with each inlet port and connecting each associated inlet port to said microchannel; and
an inlet valve mechanism associated with each inlet port.

27. A method of controlling fluid in a microfluidic device comprising the steps of:
passing at least one reactive fluid through at least one microfluidic feeder channel;
passing at least one buffer fluid through at least one microfluidic buffer channel, wherein said at least one microfluidic feeder channel and said at least one microfluidic buffer channel are in fluid communication with each other and a main microfluidic channel;
reversing a direction of flow of said at least one microfluidic feeder channel using a negative pressure differential between said feeder channel and said buffer channel; and
drawing said one buffer fluid into said at least one microfluidic feeder channel using the negative pressure differential.
